(19) United States
(12) Patent Application Publication
(10) Pub. No.: US 2010/0101572 A1
(43) Pub. Date: Apr. 29, 2010

(54) METERED DOSE INHALER FOR AEROSOL

(76) Inventor: Annamaria Rizzi, Erba (IT)

Correspondence Address: Steinfl & Bruno, 301 N Lake Ave Ste 810, Pasadena, CA, 91101, US

(21) Appl. No.: 12/532,577

(22) PCT Filed: Sep. 19, 2007
(86) PCT No.: PCT/IT2007/000652

§ 371 Date: Nov. 23, 2009

(30) Foreign Application Priority Data
Mar. 27, 2007 (IT) MI2007A 000612

(51) Int. Cl. A61M 15/00

(52) U.S. Cl. 128/203.15

(57) ABSTRACT

A metered dose inhaler for aerosol comprises an actuator (1) provided with a cavity (Id), suitable for housing a metered dose container (2) for aerosol provided with a delivery valve (2a), and with a mouthpiece (3) arranged downstream of the cavity and suitable for enabling a user to inhale a metered dose of drug delivered by the container (2). The cavity (1d) is provided with a bottom (1f) wherein a nozzle (4) is formed suitable for housingi the delivery valve (2a) of the container (2) in order to allow the delivery of a metered dose of drug, and the nozzle (4) communicates with a chamber (5) defined inside the actuator (1). The chamber (5) is tightly closed at one end by means of a first unidirectional closing valve (6) arranged in proximity to the mouthpiece (3) and at the opposite end by means of one or more second unidirectional closing valves (7) arranged in correspondence to a plurality of apertures (8) formed therein.

The present invention relates to an improved metered dose inhaler for aerosol.

Asthma is an extremely common pathology characterized by a difficult air flow through the bronchial tubes, particularly during the exhalation phase. This results in cough as well as shortness of breath for the patient.

Asthma diseases require a continuous treatment consisting in making the patient take drugs through aerosol on a regular base. In such a way, the inflammation condition of the air ducts can be kept under control without resulting in the so-called bronchial hyperactivity. The treatments for the asthma diseases last throughout the patient's life.

A type of very diffused devices for the treatment of asthma diseases are metered dose inhalers, also known under the acronym MDI. Such devices usually comprise a container under pressure, provided with a delivery valve and containing a drug in the liquid state, and an actuator provided with an inhalation mouthpiece. The container is generally vertically inserted into the actuator so that the valve enters into an axial seat suitably arranged at the bottom of the actuator. The seat housing the container valve is provided with a transversally arranged nozzle whose outlet faces the actuator mouthpiece. By pushing the container towards the inside of the actuator, the valve delivers a metered dose of drug that is deviated by the nozzle towards the mouthpiece in order to be inhaled by the patient.

Although metered dose inhalers are extremely practical, inexpensive and not very cumbersome, it is known that the percentage of drug actually reaching the bronchial tubes of a patient is only about 30% of the dose delivered by the container. This is mainly due to the fact that metered dose inhalers require a good coordination between the step of delivery of the drug from the container and the inspiratory step enabling to inhale the drug, which coordination a patient, especially a baby, can not always achieve especially during an asthma crisis. Moreover, the pressure of the spray delivered through the mouthpiece causes the drug to mainly deposit onto the oropharyngeal duct, thus reaching the bronchial tubes, which are the real target of the drug, in a lower percentage only.

It has been known for many years the use of auxiliary devices, known as “spacers”, to be applied to metered dose inhalers in order to solve these drawbacks. These devices are essentially tubes of suitable diameter and length that are inserted at one end onto the mouthpiece of the inhaler and exhibit at the opposite end a second mouthpiece, which actually enables the delivery of the drug and is provided with a unidirectional valve. By pushing on the container the drug is delivered into the spacer, where it stays until it is inhaled later, rather than into the oral duct of the patient. Thus, spacer devices eliminate the need for coordinating the inspiratory act with the step of delivery of the drug. Further, the unidirectional valve of the spacer is triggered by the patient only when a given depression threshold is exceeded, thus enabling the drug to reach the bronchial tubes more in depth. The result of using a spacer combined with a metered dose inhaler is that higher percentages of drug reach the bronchial tubes, thus improving the efficacy of the treatment and minimizing the waste of drug.

An example of spacer is given by British patent GB 2230456 to Glaxo Group Limited, wherein an inhalation chamber, particularly suited for babies, is described to be used with a metered dose inhaler for aerosol. The chamber is provided with an inlet suitable for housing the mouthpiece of a metered does inhaler and with an outlet provided with a unidirectional valve and a mask for the face. The patent underlines the importance of the volume of the spacer chamber, which has to be preferably 5 to 15 times greater than the inhalation respiratory volume of a patient in order to make the greater amount of drug as possible reach the mouthpiece of the device. The length of the spacer chamber also contributes to maximize the amount of drug reaching the mouthpiece and thus the outflow efficiency, therefore the teaching of the patent allows to correctly dimension a spacer in function of the kind of patient who will use it.

The market of aerosol products offers a complete range of sizes of spacers to be applied to a metered dose inhaler, however they have the big disadvantage of being auxiliary devices to be added to the inhaler and having to be chosen on the basis of the age and the inspiratory capacity of the patient. In addition, spacers are quite expensive and cumbersome, thus almost completely eliminating the advantages of compactness, transportability and inexpensiveness of metered dose inhalers.

Another drawback of the spacers is that they must be inserted onto the mouthpiece of metered dose inhalers, resulting in potential problems of loss of the drug delivered.

It is therefore an object of the present invention to provide an improved metered dose inhaler, which is free from said disadvantages. Said object is achieved with a metered dose inhaler, whose main features are disclosed in the first claim, while other features are disclosed in the remaining claims.

The improved metered dose inhaler according to the present invention comprises an actuator inside which a chamber functioning as a spacer is formed, receiving and retaining the drug delivered by the container under pressure. The chamber is normally tightly closed by means of a first unidirectional closing valve arranged at the end of the chamber that is in proximity to the inhalation mouthpiece, and by means of one or more second unidirectional closing valves arranged at the opposite end of the chamber in correspondence to a plurality of apertures formed into the top surface of the actuator. The opening of the first unidirectional closing valve determines the opening of said one or more second unidirectional closing valves, thus enabling an inflow of air passing throughout the chamber from one end to the other favoring the outflow of the dose of drug.

The main advantage of the inhaler according to the present invention is that, thanks to the integration of the spacer in the actuator body, a patient carries with him one single inhaler-spacer device whose size is strongly reduced with respect to the inhaler-spacer assembly of the prior art.

Consequently, the drug loss problems during the step of delivery of the drug caused by the insertion of the spacers onto the mouthpiece of the metered dose inhalers of the prior art are completely eliminated and the manufacturing costs are extremely reduced, resulting in potential competitive advantages in the market of portable devices for aerosol.

Another advantage offered by the invention is that, thanks to the opening of said one or more unidirectional valves, the volume of the chamber functioning as a spacers is virtually infinite during the inhalation step and thus capable of adapting to the inspiratory capacity of any user, being either an adult or a baby, allowing to solve the technical prejudice of being obliged to have an accumulation volume for the specific type and age of the patient.

Moreover, the air flow passing throughout the chamber from one end to the other enables the complete outflow of the dose of drug, avoiding rests to remain inside the inhaler.

Further advantages and features of the improved metered dose inhaler according to the present invention will become clear to those skilled in the art from the following detailed and non-limiting description of an embodiment thereof with reference to the attached drawings, wherein:

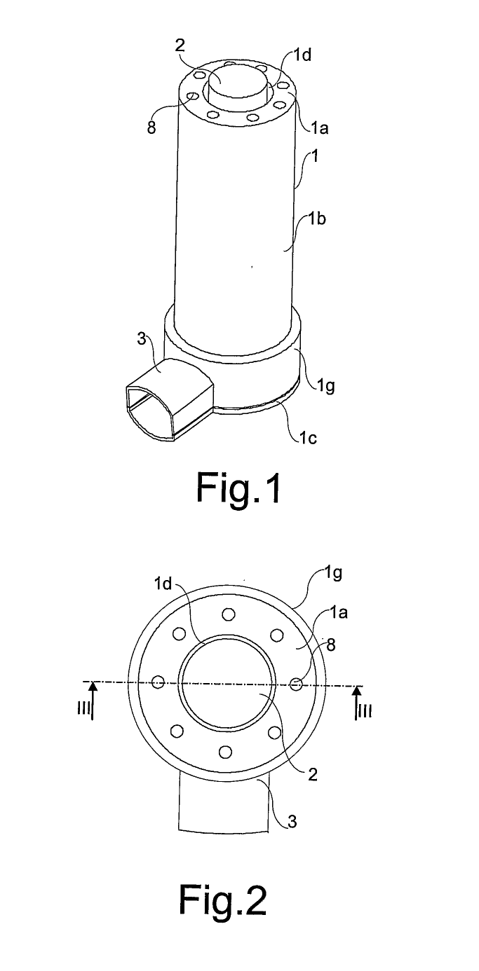

FIG. 1 shows a perspective view of an aerosol inhaler according to the present invention;

FIG. 2 shows a top view of the aerosol inhaler of FIG. 1;

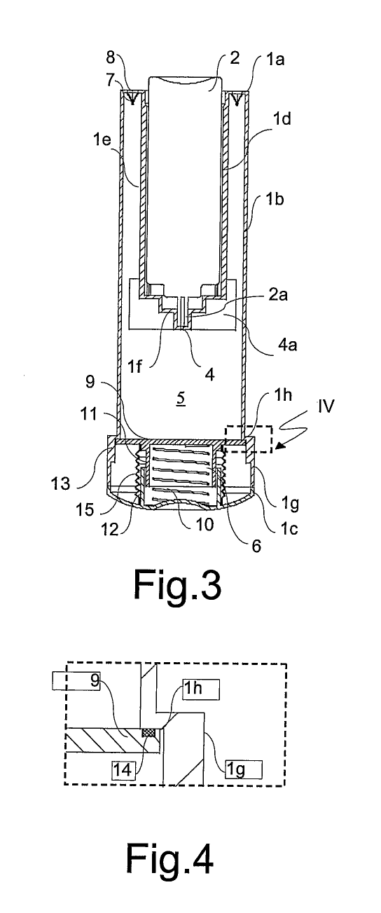

FIG. 3 shows a longitudinal sectional view along line III-III of FIG. 2;

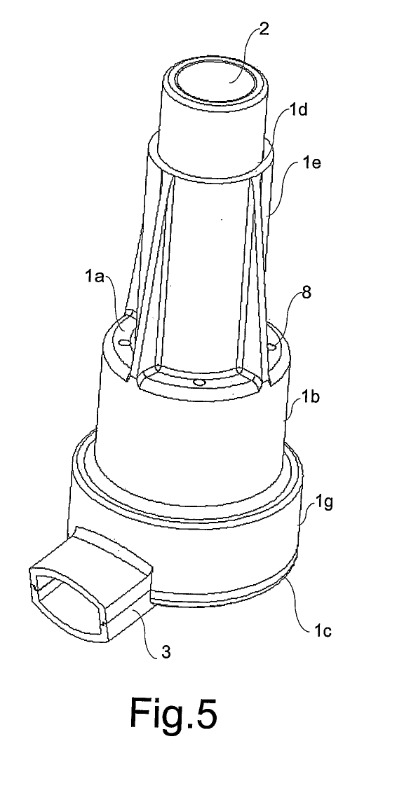

FIG. 4 shows a detail IV of FIG. 3;

FIG. 5 shows a perspective view of a preferred embodiment of the inhaler according to the invention;

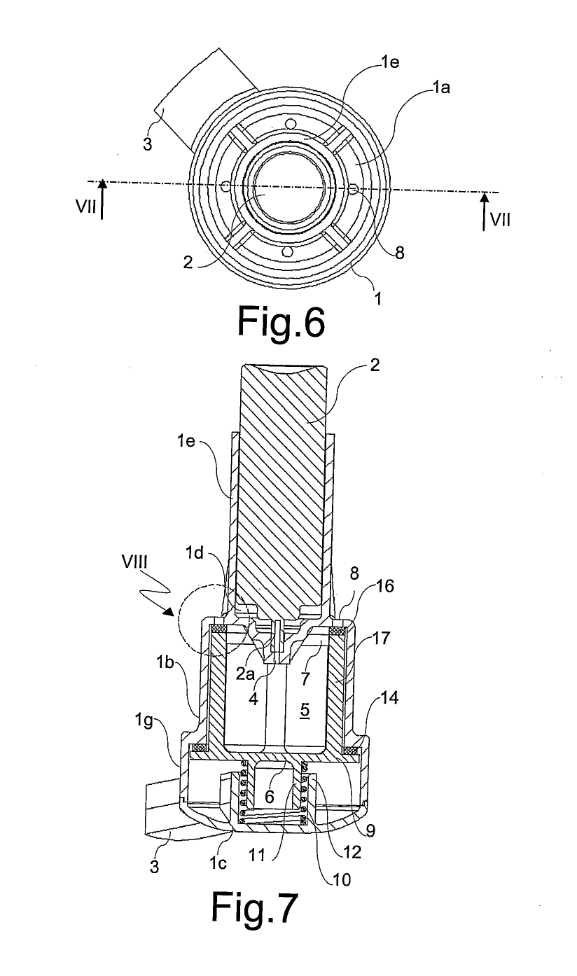

FIG. 6 shows a top view of the inhaler of FIG. 5;

FIG. 7 shows a longitudinal sectional view VII-VII of FIG. 6;

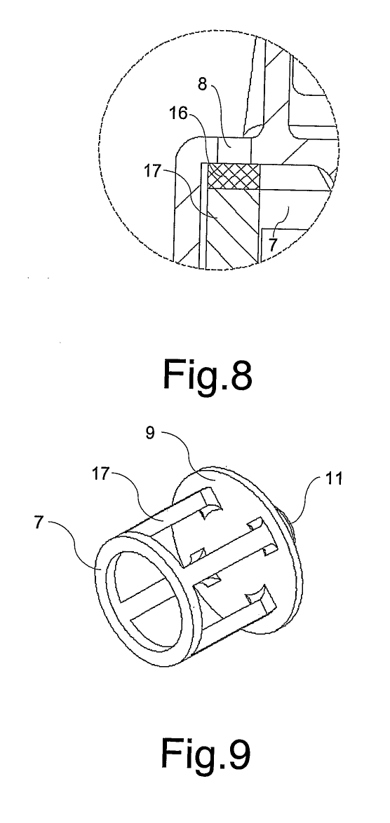

FIG. 8 shows a detail VIII of FIG. 7; and

FIG. 9 shows a perspective view of the assembly of the first and second unidirectional closing valves.

Referring to FIG. 1 and 2, the metered dose inhaler for aerosol according to the present invention comprises in a known way an actuator 1 formed of a top surface 1a, a side wall 1b and a lower lid 1c. Actuator 1 is provided with a cavity 1d suitable for housing a common metered dose container 2 for aerosol provided with a delivery valve 2a. Actuator 1 is further provided with a mouthpiece 3 arranged downstream of cavity 1d and suitable to enable a user to inhale a metered dose of drug delivered by container 2.

As shown in FIGS. 3 and 4, cavity 1d of the inhaler according to the present invention is substantially coaxially arranged relative to side wall 1b and is in turn provided with a side wall 1e and a bottom 1f wherein a nozzle 4 is formed suitable for seating delivery valve 2a of container 2 in order to allow a metered dose of drug to be delivered. Nozzle 4 communicates with a chamber 5 defined inside actuator 1 and chamber 5 is normally tightly closed at one end by means of a first unidirectional closing valve 6 arranged in proximity to mouthpiece 3 and at the opposite end by means of one or more second unidirectional closing valves 7 arranged in correspondence to one or more apertures 8 formed therein.

During the operation of the inhaler, container 2 is pressed towards the inside of actuator 1 thus delivering a metered dose of drug. The dose of drug enters chamber 5 through nozzle 4 and is kept therein by unidirectional closing valves 6, 7. Subsequently, the inspiratory act of a patient causes the opening of first unidirectional closing valve 6. This generates a depression inside chamber 5, which activates a flow of the dose of drug towards mouthpiece 3 and at the same time determines the opening of the one or more second unidirectional closing valves 7, thus generating an air flow inside chamber 5, which passes throughout it from one end to the other, i.e. from top surface 1a to first unidirectional closing valve 6, favoring the outflow of the dose of drug through mouthpiece 3.

Chamber 5 has a volume Comprised between 5 and 100 ml, necessary to grant enough space to receive the dose of drug delivered by container 2. The opening of the one or more unidirectional valves 7 makes virtually infinite the volume of chamber 5 and thus suitable to any pulmonary capacity, thus allowing to accomplish one single size of inhaler for any kind of patent.

Cavity 1d is preferably connected to side wall 1b of actuator 1 by means of a plurality of radial ribs 4a suitable or withstanding the compression force of container 2 needed for triggering valve 2a during the delivery of a dose of drug. Preferably, radial ribs 4a are arranged in correspondence to bottom 1f of cavity 1d.

In proximity to mouthpiece 3 actuator 1 is provided with a portion 1g of larger diameter that houses first unidirectional closing valve 6. First unidirectional closing valve 6 comprises a plate 9 arranged in abutment on a shoulder 1h defined at larger diameter portion 1g of actuator 1. Plate 9 is pressed on shoulder 1h by means of a spring 10 arranged between lower lid 1c and plate 9 itself.

In order to allow a correct opening and closing movement in the axial direction of first unidirectional closing valve 6, plate 9 is provided with a hollow stem 11 wherein spring 10 is inserted, and hollow stem 11 is in turn slidably inserted in a cylindrical sleeve 12 integral with lower lid 1c.

In addition, actuator 1 preferably comprises a series of longitudinal ribs 13 arranged on the inner periphery of larger diameter portion 1g, which help in guiding plate 9 along the axial direction.

In order to ensure a good sealing action of the drug inside chamber 5, an annular sealing element 14, e.g. made of silicon rubber, is preferably arranged between plate 9 and shoulder 1h.

In order to prevent the drug to meander between hollow stem 11 and cylindrical sleeve 12, a protection element 15 is provided, which is tightly inserted with one of its ends on hollow stem 11 of plate 9 and with the other end on cylindrical sleeve 12 of lower lid 1c. In order to follow the opening and closing movements of first unidirectional closing valve 6, protection element 15 may be, for instance, a flexible bellows element.

In the embodiment shown in FIGS. 1 to 4, a plurality of second unidirectional closing valves 7 are shown, e.g. of a lamellar type, being each arranged in correspondence to an aperture 8 and opening towards the inside of chamber 5 upon the depression generated in the chamber at the opening of first unidirectional valve 6.

Alternatively, it is possible to provide second unidirectional closing valves 7 by means of a single annular membrane tightly fixed on apertures 8 and provided with a series of lip slits, being each arranged is correspondence to an aperture 8, which, similarly to lamellar valves, open towards the inside of chamber 5 upon the depression generated in the chamber at the opening of first unidirectional valve 6.

FIGS. 5 to 8 show a referred embodiment of the metered dose inhaler for aerosol according to the present invention, wherein the structure of actuator 1 has been optimized in view of manufacturing needs. Top surface la is now at the base of side wall 1e surrounding cavity 1d, thus clearly separating the seat of container 2 from chamber 5. Side wall 1e has been suitably stiffened by means of a series of radial ribs.

According to this embodiment, as shown in FIGS. 7 and 8, apertures 8 are closed by means of a single second unidirectional closing valve 7, having an annular shape and being provided with a sealing element 16, e.g. a silicon ring, arranged on the surface contacting apertures 8.

Second unidirectional closing valve 7 is rigidly connected in the axial direction to plate 9 of first unidirectional closing valve 6 through a series of mountings 17. Thus, upon the inspiratory act of a patient, the movement of first closing valve 6 will immediately determine the movement of second closing valve 7, resulting in the opening of chamber 5 in correspondence to its ends at the same time in order to enable the outflow of the drug. This solution is particularly advantageous as the absence of a time delay in the opening of the second closing valve with respect to the first one allows to immediately reach the ideal outflow conditions for the drug.

Possible modifications and/or additions may be made by those skilled in the art to the hereinabove disclosed and illustrated embodiments while remaining within the scope of the following claims.

1-8. (canceled)

9. A metered dose inhaler comprising:
an actuator provided with a cavity and a mouthpiece arranged downstream of the cavity;
a chamber inside the actuator, the chamber comprising a plurality of apertures,
a nozzle inside the actuator connecting the cavity with the chamber;
a first unidirectional closing valve comprising a plate, the first unidirectional closing valve being arranged in proximity of the mouthpiece and tightly closing the chamber at one end thereof;
one or more second unidirectional closing valves arranged in correspondence of the plurality of apertures; and
a plurality of mountings rigidly connected with the one or more second unidirectional closing valves and the plate in an axial direction of the inhaler.

10. The inhaler according to claim 9, wherein the chamber has a volume comprised between 5 and 100 ml.

11. The inhaler according to claim 9, wherein the actuator is provided with an enlarged portion in proximity of the mouthpiece, the enlarged portion housing the first unidirectional closing valve.

12. The inhaler according to claim 11, wherein the enlarged portion comprises a shoulder, the plate being arranged in abutment with the shoulder.

13. The inhaler according to claim 12, wherein the actuator comprises:
